(19) United States
(12) Patent Application Publication
(10) Pub. No.: US 2026/0224757 A1
(43) Pub. Date: Aug. 6, 2026

(54) Echogenic Encapsulations

(71) Applicant: Brian Michael Coyle, Canyon, CA (US)

(72) Inventors: Brian Michael Coyle, Canyon, CA (US); Alys Larsen, Canyon, CA (US)

(21) Appl. No.: 19/046,529

(22) Filed: Feb. 6, 2025

(51) Int. Cl. A61K 49/22

(52) U.S. Cl. CPC A61K 49/223

(57) ABSTRACT

Echogenic Encapsulation units, components, and associated systems and methods are described. Echogenic Encapsulations can be incorporated in objects inserted into animal bodies, thereby making the objects detectable with ultrasound tools. Echogenic Encapsulations are sealed cavities with a surface transparent to ultrasound energy, containing pluralities of echogenic microbubbles and/or particles suspended in carrier fluidic substances. The echogenic microbubbles and/or particles are protected from the environment in the sealed cavity, and being so protected do not dissipate but remain functional for extended periods. Echogenic Encapsulations may be attached to object surfaces, or be positioned on subcomponents in objects.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

Acoustical detection systems for internal medicine may use spheres less than 100 μm in diameter, generally called microbubbles or gas-containing particles, that have an external shell made of a natural or synthetic polymer, lipid, protein, or other materials, including stabilizing surfactants, and contain gas, fluid, or other substance inside. Microbubbles are typically administered into an area in a patient, then activated by ultrasound (US) sources directed at the area by a medical provider outside the patient. Microbubbles may flow within a constrained circulatory or endocrine system, or associate with an organ or tissue (including embryonic) membrane.

Acoustic activation by US compresses microbubbles, producing patterns of backscattered signals that distinguish their presence. Acoustic activation permits an image of the system or associated structure to be safely produced for medical providers.

Existing coatings for some medical tools claim to be echogenic. These include polymers that contain small spherical holes, which may increase the polymer's resonance in response to US.

Others cover an object surface with micro-holes, and push a gas into the micro-holes as they are coated with a polymer, which may increase the surface echnogenicity. Polymers may be formed with gas bubbles in their matrix. These echogenic coatings are applied to surgical equipment such as surgical needles. Evidence suggests they may incrementally increase US detection of a needle that has been inserted in a body (Hovgesen, et al. 1.) U.S. Pat. No. 5,289,831 to Bosley (P1) describes echogenic medical devices having a surface with partially spherical indentations; U.S. Pat. No. 5,081,997 to Bosley et al. (P2) describes echogenic medical devices having a surface coated with spherically shaped particles. U.S. Pat. No. 6,106,473 to Violante (P3) describes a method to coat the surface of a medical object, in which a liquid substance is applied to a substrate, the liquid substance having a constituent that forms a thin film upon evaporation of the liquid during an application process, the thin film forming structures that may be echogenic during the application process. Echogenic structures may be open pores or channels that trap air at the surface of the coating, closed bubbles in a polymer matrix, or gas-filled solid inclusions precipitated during the application process. Because the size and concentrations of microbubbles formed during a coating process are difficult to control, U.S. Pat. No. 9,681,852 to Vriezema et al. (P4) teaches a coating for a medical device made of microparticles responsive to US. The microparticles are solid, formed of semi-hard or hard materials, or hollow, formed of hard materials containing gasses, and are present on the coating surface.

A number of patents similar to '852, such as U.S. Pat. No. 10,245,356 to Ayres et al. (P5) and U.S. Pat. No. 10,166,005 to Vriezema et al. (P6) describe methods of application that include a series of surface coatings, with one of the surfaces being largely composed of microparticles. For example '005 teaches that at least 60% of the surface of the coated device is to be covered with a single layer of microparticles. A single or double layer of microparticles is said to reduce surface thickness and roughness, so that a surgeon needs less force to move the coated device, such as a needle, in a patient's body. The coating of a device is accomplished by dip coating, spray coating, pad printing, roller coating, printing, painting or inkjet printing. These methods, and their use in '852, '356, and '005, produce thin film matrices in which microparticles are immobilized.

Microbubbles that are injected into blood or tissue, for US enhancement, are not immobilized in solid or semi-solid matrices like configurations for coating medical devices, but are formed as echopharmaceutical suspensions. The suspensions described more than 35 years ago in U.S. Pat. No. 4,681,119 to Rasor & Tickner (P7) were methods that generated microbubbles shortly prior to use. Gas is present in a liquid with microbubble precursor material, and microbubbles form and are maintained for up to 10 minutes. Over the next decades efforts were made to increase the duration of microbubble formations. Perfluorinated gases, such as perfluorocarbons, possess lower solubility in aqueous media than many gasses or air, and are retained inside the microbubbles in the bloodstream longer. U.S. Pat. No. 5,701,899 to Porter (P8) teaches microbubbles with internal atmospheres containing perfluorobutane gas. It has a circulation time after bodily injection of about 60 seconds, after which sonication is not detectable (Bing, et al. 2.) Microbubble suspensions are administered by intravenous injection of 1-2 mL; their concentration in blood is around 105 to 106 microbubbles/mL, which produces strong contrast enhancement even at reduced ultrasound levels (1 Wcm). All microbubbles clinically available are shipped and/or stored in sealed vials in concentrations of about 108 to 109 microbubbles/mL.

Despite the fact that microbubble suspensions remain in circulation for only a few minutes at most, in sealed vials they have a shelf life of many weeks or months (Stride, et al. 3.) Microbubbles for injection as contrast agents are typically shipped in 1 to 5 ml vials, as powder-gas mixtures reconstituted with water or other dilutent such as sodium chloride, or as a liquid with a polymeric surface layer or headspace gas that must be mixed prior to use (Sridharan, et al. 4.) Once reconstituted, microbubbles remain intact up to four weeks (without loss of acoustic contrast power) when stored in sealed vials, at 4 to 25° C. (Ojha, et al. 5.) The echogenicity of immobilized microbubbles or microbubble-like surface indentations, such as those that decorate surgical tools, vanishes as a tool is inserted more than a few cm into a patient (Hopkins & Bradley 6.) While there may be many medical practices that involve inserting tools into a patient no more than this amount, many other medical devices are positioned far deeper in a patient's body. A large human has a torso radius of ~30 cm. To use US to visualize medical devices passing through a gastrointestinal (GI) tract, or monitor medical implants, the use of immobilized microbubbles or microbubble-like surface indentations are inadequate.

For the purposes of visualizing objects inserted into a body there is a need to harness the superior echogenicity of microbubbles in suspension. However only microbubbles sealed from the body's environment can be maintained if used for medical devices that pass through the GI tract or are implanted in a body, which may need US visualization for hours, days, weeks, or longer.

US is, by far, the most used medical imaging system in the world. US transducers are widespread and portable, US methodology is safe, and US operation is inexpensive. Unlike x-ray radiography, US involves non-ionizing wavelengths. It does not risk the induction of genetic mutations (Wang, Hossack & Klibanov 7.)

Deployment of medical devices in the body, that may be visualized with US, is desirable, given they may not currently be visualized, or only with x-rays.

### SUMMARY OF THE INVENTION

An embodiment of this invention features an Echogenic Encapsulation as a component part of an object inserted into an animal body, including at least one sealed cavity that contains a plurality of microbubbles or particles concentrated in a carrier fluidic substrate, the carrier fluidic substrate stabilizing the plurality of microbubbles or particles against collapse or aggregation, the plurality of microbubbles or particles being echogenic, the at least one sealed cavity forming an area that is exposed to US radiation, with at least one outer layer of the sealed cavity exposed to US radiation being transparent to US radiation, the Echogenic Encapsulation isolating the plurality of microbubbles or particles from an external environment.

An embodiment of this invention features a method of forming an Echogenic Encapsulation that surrounds at least a part of an object, the object to be inserted in a body, the invention providing at least one layer attached to the part of the object, the at least one layer forming a sealed cavity, injecting a fluid in the sealed cavity, disposing a plurality of microbubbles or particles containing gas into the fluid, at least a part of the at least one layer being transparent to US radiation, the fluid stabilizing the plurality of microbubbles or particles against collapse or aggregation, and the plurality of microbubbles or particles having an echogenic response to US radiation.

A method of manufacturing an Echogenic Encapsulation according to another aspect of the present invention includes attaching a first layer made of a metal, plastic, or glass material to a surface of an object to be inserted in an animal body, attaching a second layer above the first layer, spaced apart from the first layer and forming a cavity between them, at least a part of the second layer being transparent to US radiation, injecting a fluid between the first layer and the second layer which is contained in the cavity, the fluid formed to include a plurality of echogenic microbubbles or particles, and attaching the first layer and second layer to seal the cavity.

An embodiment of this invention features an Echogenic Encapsulation, including at least one sealed cavity that is a component of an object to be inserted inside a body, a plurality of microbubbles or particles are concentrated in a carrier fluid in the at least one sealed cavity, the carrier fluid stabilizing the plurality of microbubbles or particles against collapse or aggregation, the plurality of microbubbles or particles being echogenic, the at least one sealed cavity formed with a surface layer that is transparent to US radiation.

An embodiment of this invention features an Echogenic Encapsulation in which the at least one sealed cavity is formed with a plurality of upstanding pillars arranged to support a laminar surface.

Embodiments of the present invention allow for increased scattering of ultrasonic energy, which increases echogenicity, as the Echogenic Encapsulation is composed of at least one sealed cavity that contains a distribution of a plurality of microbubbles or particles in a manner that the microbubbles or particles have a similar size, 5 μm in diameter, 2-5 μm in diameter, 4-6 μm in diameter, 1-8 μm in diameter, 1-20 μm in diameter, or another size distribution that increases echogenicity, including other distributions of the microbubbles or particles with a size of less than a single micrometer; the at least one sealed cavity allows a close packing of the microbubbles or particles which increases the brightness of an echogenic signal and thereby increases echogenicity; the close packing of the microbubbles or particles increases their stability which allows for prolonged US imaging.

Embodiments of the present invention may be prepared in a temporal window close enough to an insertion of an object including an Echogenic Encapsulation into a body to ensure concentrations of at least one stabilizer in the carrier fluid is not diminished and the microbubbles or particles do not collapse or aggregate.

Embodiments of the present invention are prepared with the plurality of microbubbles or particles suspended in a carrier fluid sealed in the at least one cavity to ensure concentrations of at least one stabilizer in the carrier fluid is not diminished and the microbubbles or particles do not collapse or aggregate.

Embodiments of the present invention distribute microbubbles or particles in the carrier fluid at concentrations of greater than 108 microbubbles/mL. Other embodiments of the present invention distribute microbubbles or particles in the carrier fluid at concentrations between 105 and 108 microbubbles/mL. Other embodiments of the present invention distribute microbubbles or particles in the carrier fluid at concentrations below 105 microbubbles/mL.

In an embodiment, a sandwich-like configuration of a first and a second surface is formed, the first surface a material less than 2 mm thick, such as borosilicate, and the second surface beneath the first surface, the second surface may be the outer surface of an object inserted in, or passing through, a body, with a closed cavity less than 1 mm wide formed between the first surface and the second surface. The closed cavity contains a plurality of echogenic microbubbles or particles suspended in a carrier fluid.

Embodiments of the present invention use a modular configuration of an Echogenic Encapsulation, wherein a sealed cavity that contains a plurality of echogenic microbubbles or particles, suspended in a carrier fluid, is positioned as a module on, around, or within an object

Embodiments of the present invention use carrier fluids that incorporate phospholipid as a stabilizer of the microbubbles or particles against collapse or aggregation. The concentration of phospholipids in the carrier liquid is below 0.01% by weight.

Embodiments of the present invention use carrier fluids that incorporate water soluble polysaccharides and oligosaccharides as a stabilizer of microbubbles or particles against collapse or aggregation.

Embodiments of the present invention use carrier fluids that incorporate hydrophilic polymers as a stabilizer of microbubbles or particles against collapse or aggregation.

Embodiments of the present invention use carrier fluids that incorporate nonionic surfactants as a stabilizer of microbubbles or particles against collapse or aggregation.

Embodiments of the present invention use carrier fluids that incorporate fats, waxes and hydrocarbons as a stabilizer of microbubbles or particles against collapse or aggregation.

In an embodiment, microbubbles or particles may form an inherent barrier to porosity and structural damage of an object.

In an embodiment, microbubbles or particles may be activated to release an agent contained in an object for a therapeutic or functional purpose.

Particles may be microparticles greater than 1 μm or nanoparticles 1 μm or smaller, that are solid or liquid, not gas-filled. In an embodiment particles are formed and mixed with microbubbles. In an embodiment, a mixture of particles and microbubbles increases microbubble stability. In an embodiment, mixture of particles and microbubbles form particle-microbubble hybrids.

A fluidic substrate or carrier fluidic substrate is a continuous phase fluid that is a liquid or gas that occupies a connected region of space and can contain dispersed phases. Dispersed phases can be microbubbles, particles, or droplets. Further details of these and other embodiments and aspects of the invention are described more fully below with reference to the accompanying figures.

### BRIEF DESCRIPTION OF THE FIGURES

The present invention will be understood and appreciated more fully from the following detailed description taken in conjunction with the drawings in which:

FIG. 1 is a longitudinal cross-sectional view illustrating assembled components of an Echogenic Encapsulation.

FIG. 2 is a longitudinal cross-sectional view illustrating delivery of echogenic microbubbles or particles into the Echogenic Encapsulation.

FIG. 3 is a perspective view of an object surrounded by an Echogenic Encapsulation.

FIG. 4 is a cross-sectional view of an Echogenic Encapsulation.

FIG. 5 are perspective views of Echogenic Encapsulations incorporated in a medical implant.

FIG. 6 is a cross-sectional perspective view of an Echogenic Encapsulation surrounding an internal component of an object.

FIG. 7 is a cross-sectional perspective view of an Echogenic Encapsulation positioned in an object.

FIG. 8 is an illustration of a use case for a device configured with an Echogenic Encapsulation.

### DETAILED DESCRIPTION

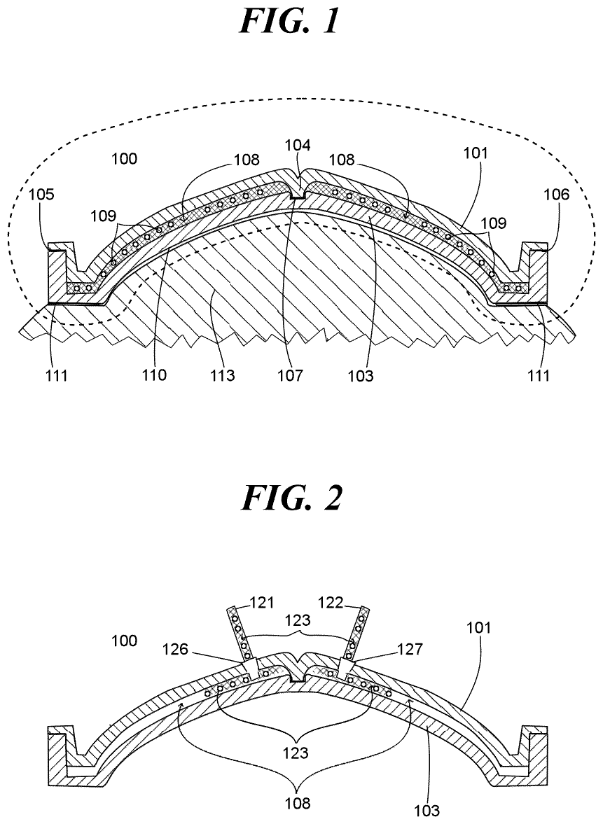

With reference to FIG. 1, an Echogenic Encapsulation 100 is formed with an outer circumferential layer 101 largely transparent to US radiation, opposite to an inner circumferential layer 103, and layer 101's inner surface and layer 103's outer surface are relatively separated from each other over most of their surfaces, and a plurality of portions 105 and 106 of layer 101's inner surface are pressed to the layer 103's outer surface where they meet, and a portion 104 of layer 101's inner surface that extends across layer 103 is fitted in an engagement slit 107 of layer 103, to form a plurality of sealed cavities 108 which contain a plurality of echogenic microbubbles or particles suspended in a fluid 109. The inner surface 110 of layer 103 is pressed into an object 113 and attached to object 113 at a plurality of bonding sites 111.

With reference to FIG. 2, the Echogenic Encapsulation 100 obtained by juxtaposition of the outer circumferential layer 101 and the inner circumferential layer 103 is illustrated to show how the plurality of sealed cavities 108, which may be in the shape of channels, are provided with the plurality of echogenic microbubbles or particles suspended in a fluid 123. A plurality of injection members 121 and 122 are each inserted into a respective orfice 126 and 127, through which the fluid containing microbubbles or particles 123 is injected into the plurality of sealed cavities 108.

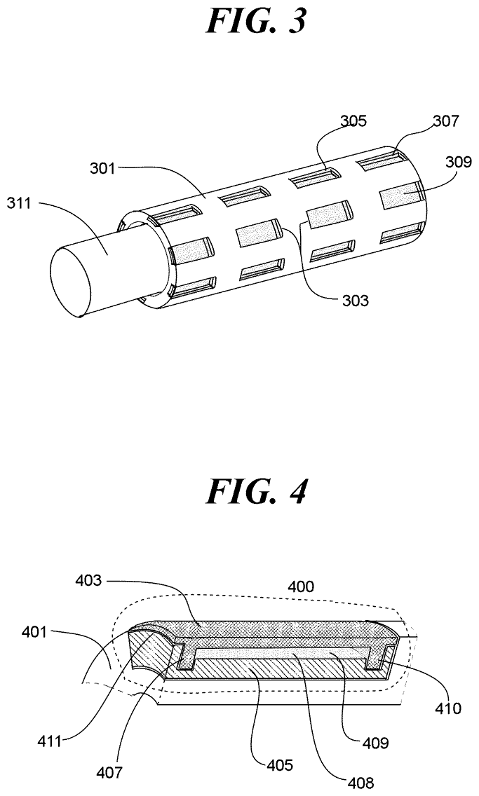

Referring to FIG. 3, a plurality of Echogenic Encapsulation units, each the form of a concavity 303 that contains echogenic microbubbles or particles suspended in a fluid 309, are provided in a circumferential layer of a cylindrical housing 301, each Echogenic Encapsulation unit 303 having an arcuate shape with an outside layer 305 transparent to US radiation, and a sealed cavity 307 that contains echogenic microbubbles or particles suspended in a fluid 309, the cyclindrical housing 301 covering an object 311 fitted and retained in the housing 301, and as illustrated in FIG. 3 the object 311 is partially withdrawn from the housing 301.

Referring to FIG. 4 an arcuate Echogenic Encapsulation unit 400 is illustrated, the outside layer 403 transparent to US radiation being separated from the inside layer 405 except where they contact at locations such as 407, 410, and 411 to form sealed cavity 409 that contains echogenic microbubbles or particles suspended in a fluid 408, the Echogenic Encapsulation unit 400 abutting and attached to a peripheral portion of the cylindrical housing 401.

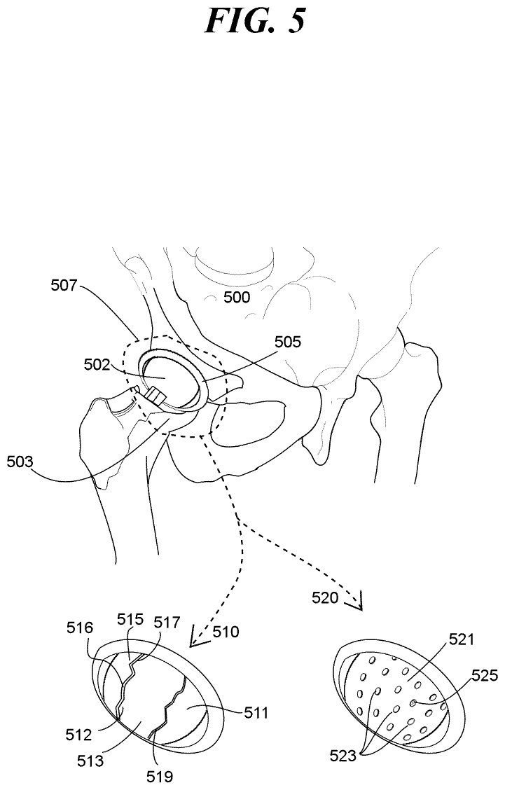

FIG. 5 illustrates an anatomy 500 of a patient having a ceramic or metal ball 502 inserted into a hip area 503 as part of a hip prosthesis to replace a damaged femoral head. The ball 502 and surrounding sidewalls 505 together 507 are shown in enlarged views 510 and 520. Enlarged view 510 includes a continuous coating layer 511 applied to the ball 513, the surface layer 511 shown in cutaway 512 having a sealed cavity 516 between an outer surface 515 and inner surface 517, the sealed cavity 516 containing echogenic microbubbles or particles suspended in a fluid 519.

Referring as well to enlarged view 520, the coating layer 521 is characterized by a distribution of Echogenic Encapsulation units 523, each containing echogenic microbubbles or particles suspended in a fluid 525.

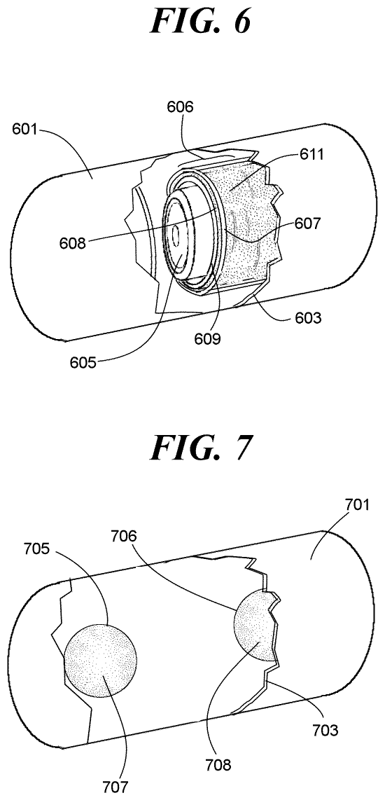

Referring to FIG. 6 a swallowable medical device capsule 601 is shown with a cutaway 603 that reveals a battery unit 605 inside, which is surrounded by an Echogenic Encapsulation unit 606 composed of an outer layer 607 transparent to US radiation, an inner layer 609, and a cavity between them 608 that contains echogenic microbubbles or particles suspended in a fluid 611.

Referring to FIG. 7 a swallowable medical device capsule 701 is shown with a cutaway 703 that reveals internal sphere 705 and internal sphere 706, each with an Echogenic Encapsulation surface 707 and 708 that extends across their surfaces. This allows the position of the device 701 to be detected with US at any orientation in a body.

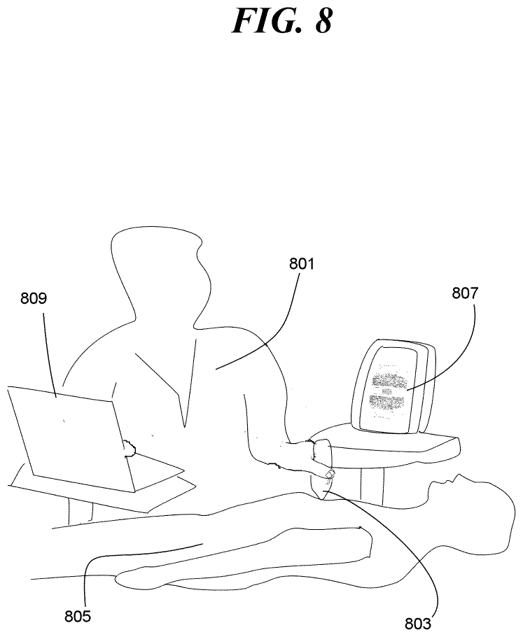

FIG. 8 illustrates a use case for Echogenic Encapsulations, in which a medical provider 801 uses a US monitoring device 803 to track a medical device outfitted with an Echogenic Encapsulation as it moves through a patient 805, the US signal visible on a display 807, which the medical provider 801 refers to in determining whether to modify the medical device trajectory using a medical device interface 809.

Embodiments of Echogenic Encapsulations may be configured for other animals than humans, and for structures in the body besides capsules and prosthesis, including temporary structures used for surgical and post-surgical purposes, for dental and ear devices, as well as other medical structures.

The term “microbubble” refers to a micrometer-sized object with a shell and a gas core.

Shell materials like proteins, lipids, and polymers disperse in an aqueous solution with a surfactant, or a combination of two substances can be used for stability.

The term “particle” refers to stable gas-filled microbubbles with solid material shells.

Particles remain stable in an aqueous media for years. They are not biodegradable, and do not exit the body. Since current technology injects microbubbles that freely circulate in a body vessel or area, particles are not widely used in medical imagining. However in this invention particles may be used, as they remain isolated from the body environment in an Echnogenic Encapsulation.

The term “US” refers to diagnostic frequencies in the range from 1 and 50 MHz. Lower frequencies have greater depth of tissue penetration (>5 cm) and are preferred for detecting objects in a GI tract or in deep tissues.

The term “sealed cavity” refers to an area that does not exchange fluid, gas, or particles with an external environment. The term “echogenic” means something that reflects sound waves, or echoes, and appears bright on an ultrasound. The term “encapsulation” refers to an enclosure that contains something.

“At least one of A and B” should be understood to mean “only A, only B, or both A and B.” “Selected from the group of A, B, and C” should be understood to mean “only A, only B, only C, or both A and B, or both A and C, or both B and C, or A, B, and C.” “At least one selected from one or more of A, B, C, and D” should be understood to mean “only A, only B, only C, only D, or both A and B, or both A and C, or both A and D, or both B and C, or both B and D, or both C and D, or A, B, and C, or A, B, and D, or A, C, and D, or B, C, and D, or A, B, C, and D.”

As used herein, an element or step recited in the singular and proceeded with the word “a” or “an” does not exclude plural of said elements or steps, unless such exclusion is explicitly stated.

References to an “embodiment” do not exclude the existence of additional embodiments that also incorporate the recited features. Embodiments “comprising,” “including,” or “having” an element (component, part) or a plurality of elements (components, parts) having a particular property may include additional such elements not having that property.

It will be appreciated by persons skilled in the art that the present invention is not limited to what has been particularly shown and described herein above. Rather the scope of the present invention is defined only by the claims which follow.

1. An Echogenic Encapsulation for use as part of an object inserted into an animal body, the Echogenic Encapsulation comprising:
at least one or more surface;
one or more layers configured to cover the at least one surface;
wherein the one or more layers form at least one sealed cavity that contains a fluidic substrate;
a plurality of echogenic microbubbles or particles are stabilized in the fluidic substrate;
at least one of the one or more layers is transparent to ultrasonic energy.

2. The Echogenic Encapsulation of claim 1, wherein:
the at least one surface includes at least one channel
the at least one of the one or more layers seals the fluidic substrate in the at least one channel.

3. The Echogenic Encapsulation of claim 1, wherein:
the at least one surface includes at least one concavity,
the at least one of the one or more layers seals the fluidic substrate in the at least one concavity.

4. The Echogenic Encapsulation of claim 1, wherein:
the at least one sealed cavity is formed with a plurality of upstanding pillars arranged to support at least one of the one or more layers.

5. The Echogenic Encapsulation of claim 1, wherein:
the microbubbles or particles have a similar size, the similar size may be less than a single μm to greater than 20 μm.

6. The Echogenic Encapsulation of claim 1, wherein:
the microbubbles or particles are closely packed in the at least one sealed cavity.

7. The Echogenic Encapsulation of claim 1, wherein:
the fluidic substrate is acoustically homogeneous.

8. The Echogenic Encapsulation of claim 1, wherein:
at least two of the one or more layers are separated by a distance of <2 millimeters.

9. The Echogenic Encapsulation of claim 1, wherein:
a medical provider uses an ultrasound tool to interrogate the Echogenic Encapsulation.

10. The Echogenic Encapsulation of claim 1, wherein the Echogenic Encapsulation is assembled less than 24 hours prior to being inserted into the animal body.

11. A method of making an Echogenic Encapsulation to be inserted into an animal body,
assembling a plurality of layers, including at least one first layer and at least one second layer,
at least one of the plurality of layers is transparent to ultrasonic energy,
sealing at least one of the first layers and at least one of the second layers to form a cavity,
injecting a carrier fluidic substance including a plurality of echogenic microbubbles or particles into the cavity,
ensuring the plurality of echogenic microbubbles or particles do not dissipate when the Echogenic Encapsulation is inserted in the animal body.

12. The method of claim 11, wherein the injection of the carrier fluidic substance including the plurality of echogenic microbubbles or particles is through at least one valve in at least one of the plurality of layers.

13. The method of claim 11, wherein the plurality of layers form a plurality of cavities. microbubbles or particles are injected in each of at least two of the plurality of cavities.

15. The method of claim 11, wherein the carrier fluidic substance stabilizes the plurality of echogenic microbubbles or particles.

16. An Echogenic Encapsulation installed in an object inserted into an animal body, the Echogenic Encapsulation comprising:
at least one sealed cavity;
the at least one sealed cavity being formed by a first layer and at least a second layer;
at least one of the first layer and the at least second layer being transparent to ultrasonic energy;
the at least one sealed cavity being defined by a space between the first layer and the at least second layer;
the at least one sealed cavity containing a carrier fluidic substance;
the at least one sealed cavity being protected from an external environment;
at least one valve in at least one of the first layer and the at least second layer;
the carrier fluidic substance being introduced through the at least one valve;
wherein the carrier fluidic substance includes a plurality of echogenic microbubbles, a plurality of echogenic particles, or both; and
wherein the Echogenic Encapsulation enables ultrasound detection of the object.

17. The Echogenic Encapsulation of claim 16, wherein the first layer is an upper layer above the at least second layer, the first layer being transparent to ultrasonic energy, and the first layer and the at least second layer being connected by at least one side portion

18. The Echogenic Encapsulation of claim 16, wherein the Echogenic Encapsulation is installed inside the object.

19. The Echogenic Encapsulation of claim 16, wherein the Echogenic Encapsulation is installed on an external surface of the object.

20. The Echogenic Encapsulation of claim 16, wherein the plurality of echogenic microbubbles and the plurality of echogenic particles are mixed together.
